(19) United States
(12) Patent Application Publication
(10) Pub. No.: US 2009/0292284 A1
(43) Pub. Date: Nov. 26, 2009

(54) FLEXIBLE SHAFT DRIVING ATTACHMENT FOR TOOLS

(76) Inventors: Serafino CANINO, Torrington, CT (US); Scott Gelormino, Torrington, CT (US)

Correspondence Address: THOMPSON HINE L.L.P.;Intellectual Property Group, P.O. BOX 8801, DAYTON, OH, 45401-8801, US

(21) Appl. No.: 12/124,601

(22) Filed: May 21, 2008

(51) Int. Cl. A61B 17/56; B21J 15/28

(52) U.S. Cl. 606/53; 227/8

(57) ABSTRACT

A flexible extension shaft comprises a flexible extension member having a length, and one end and an opposite end. A tool base connection member is secured to one end of the flexible extension member. A tool bit connection member is secured to the opposite end of the flexible extension member. A lighting unit is secured to the flexible extension shaft.

### TECHNICAL FIELD

The invention relates to flexible extension shafts for extending the reach of hand tools and power tools, such as screwdrivers.

### CROSS REFERENCE TO RELATED APPLICATIONS

(Not applicable)

### STATEMENT REGARDING FEDERALLY SPONSORED RESEARCH OR DEVELOPMENT

(Not applicable)

### BACKGROUND OF THE INVENTION

Many tools, such as screwdrivers, have a chuck which receives a tool bit, thus allowing the use of the tool for a plurality of functions. For example, many screwdrivers terminate in a hexagonal chuck which simply comprises a hexagonal hole configured to receive a standard bit having a hexagonal base. Such bits are commonly available, often featuring Phillips-type screwdriver heads of various size, slot-type screwdriver heads of various size, and other tools.

Extension shafts for such tools are also well known, typically comprising a hexagonal base configured to be received in the hexagonal chuck of a standard tool. The hexagonal base is coupled to a flexible extension member which, in turn, is coupled to hexagonal chuck. The effect is to extend the reach of the tool by about the length of the flexible extension member. At the same time, the flexibility of the extension member allows a tool to perform a function in what might be otherwise an unreachable place, for example inside an item of machinery.

### SUMMARY OF THE INVENTION

In accordance with the invention, an improved flexible extension shaft featuring an illumination light is provided.

More particularly, a flexible extension shaft comprises a flexible extension member having a length, and one end and an opposite end. A tool base connection member is secured to one end of the flexible extension member. A tool bit connection member is secured to the opposite end of the flexible extension member. A lighting unit is secured to the flexible extension shaft.

The lighting unit may be slidingly mounted on the flexible extension member.

The flexible extension member may comprise an outer sleeve and a central rotatable member which rotates within the outer sleeve. The tool base connection member and the tool bit connection member may be secured to the central rotatable member.

Connection member may be included for securing the lighting unit to the flexible extension member.

The connection member may be a closed loop extending around the flexible extension member.

Alternatively, the connection member may be a clip.

The lighting unit may incorporate a switch.

The lighting unit a slide on the flexible extension member.

The tool base connection member may comprise a hexagonal bit for mating with the output of a power or hand tool and the tool bit connection member may be a hexagonal socket chuck.

### BRIEF DESCRIPTION THE DRAWINGS

The operation of the invention will become apparent from the following description taken in conjunction with the drawings, in which:

FIG. 1 is a perspective view of a flexible extension shaft in accordance with the present invention, illustrating the workpiece illuminating light positioned proximate to the work area, for example illuminating a screw being tightened by the shaft using a power or manual tool driver;

FIG. 2 is a view similar to that of FIG. 1 with the lighting unit in the rearward position;

FIG. 3 is a detailed perspective of the lighting unit positioned adjacent the workpiece;

FIG. 4 is a schematic diagram of an electrical circuit for use in a flexible extension shaft according to the present invention;

FIG. 5 is a perspective view of an illumination device useful in the inventive extension shaft with the housing cap removed to exposing internal working parts, but showing the switch and batteries in a working position;

FIG. 6 is a cross-sectional schematic illustration of the illuminating unit illustrated in Figures numerals 1-5 with the batteries removed;

FIG. 7 is a view similar to FIG. 6 with the batteries in place;

FIG. 8 is a view similar to FIG. 5 illustrating wiring of power from the switch to the light source;

FIG. 9 is a perspective view illustrating the drive receiving end of the inventive flexible extension shaft;

FIG. 10 is a view similar to FIG. 9 with the illumination unit grippingly engaged by the shaft base collar;

FIG. 11 illustrates the socket end of the flexible extension shaft;

FIG. 12 is a view similar to that of FIG. 8 illustrating an alternative embodiment of the invention which may be clipped onto an existing flexible or inflexible shaft;

FIG. 13 illustrates an alternative embodiment of the present invention using a coiled contact spring structure;

FIG. 14 illustrates the coiled contact spring structure;

FIG. 15 illustrates the embodiment of FIGS. 13 and 14 in an exploded cross-sectional, schematic view;

FIG. 16 illustrates cap of the alternative embodiment of FIG. 15 in a cross-sectional, schematic view; and

FIG. 17 illustrates base of the alternative embodiment of FIG. 15 in a cross-sectional, schematic view.

### DETAILED DESCRIPTION OF THE PREFERRED EMBODIMENTS

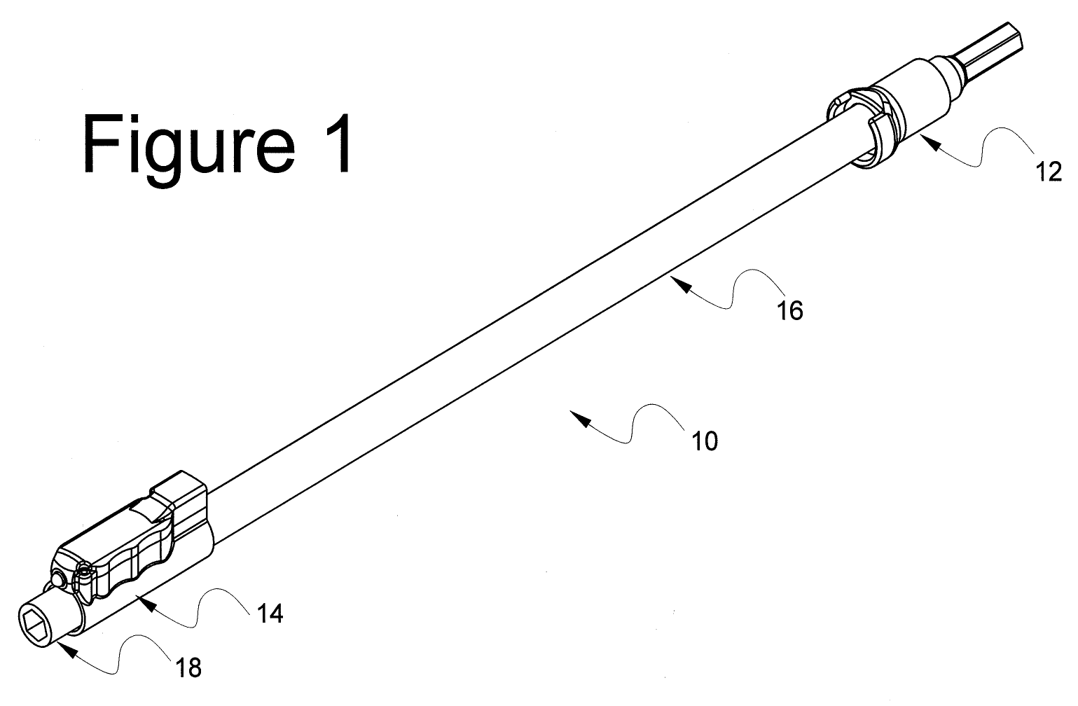

Turning to FIG. 1, an extension shaft 10 constructed in accordance with the present invention is illustrated. Extension shaft 10 comprises a hexagonal shaft base 12 and an illumination unit 14. Hexagonal shaft base 12 is secured to a flexible extension member 16. An extension shaft chuck 18 is secured to the other end of flexible extension member 16.

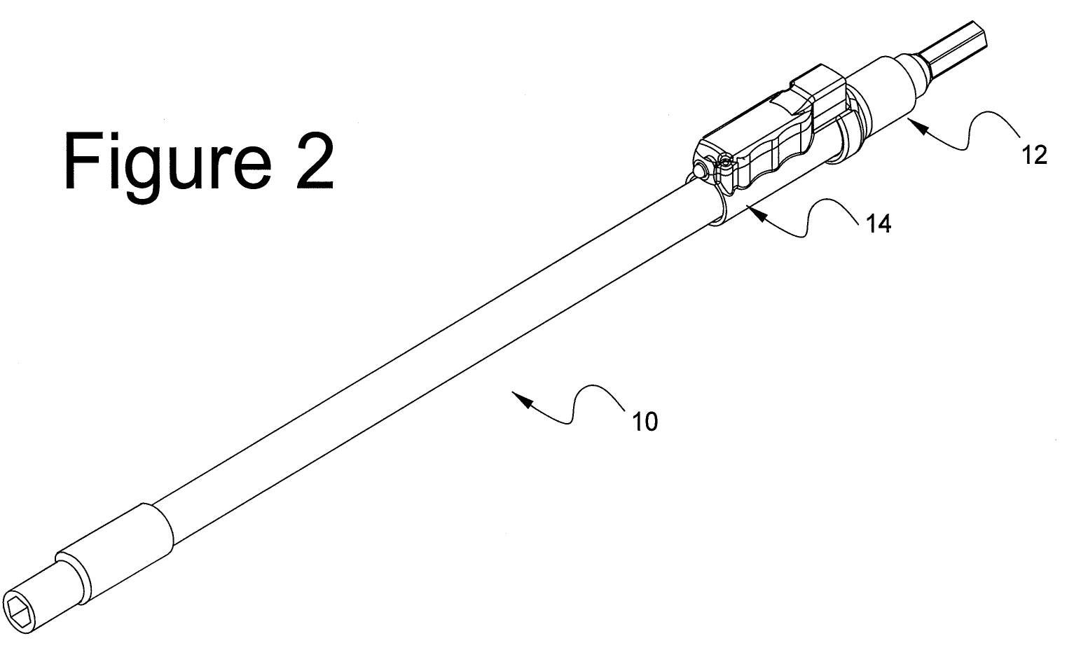

In FIG. 1, illumination unit 14 is illustrated on extension shaft 10 in the illuminating position. Optionally, illumination unit 14 may be positioned on extension member 16 of extension shaft 10 in the light storage position as illustrated in FIG. 2. Repositioning is done by simply sliding illumination unit 14 on extension member 16 until it is in the desired position, which in most cases it is the most scored position, as illustrated in FIG. 1. Alternatively, illumination unit 14 may be slid to the rearmost position illustrated in FIG. 1.

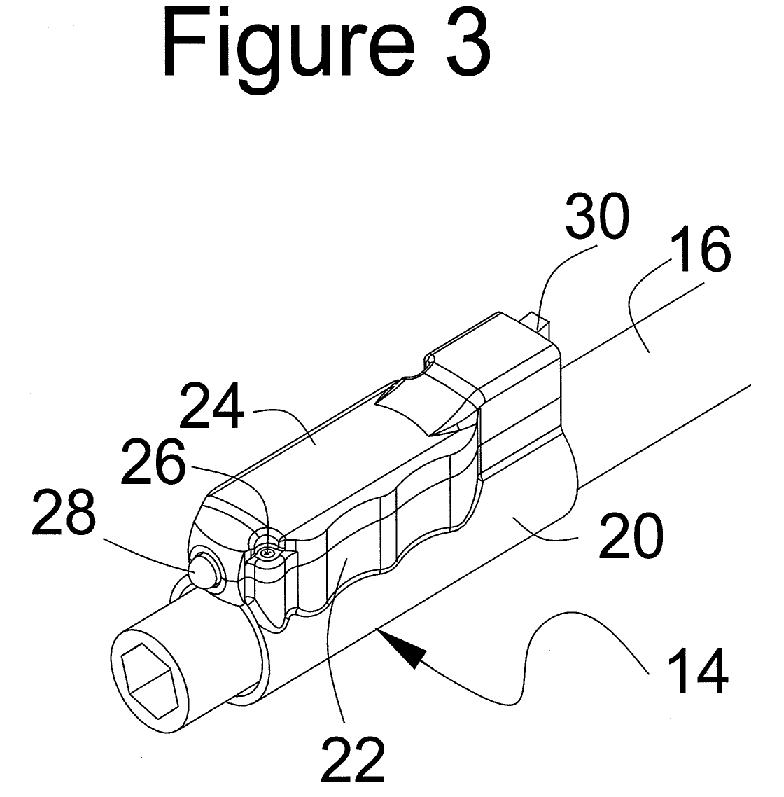

Generally, illumination unit 14 comprises a tubular sleeve 20 configured to snugly fit around extension member 16, as illustrated most clearly and FIG. 3. This enables illumination unit 14 to slide to any position along the extension member 16. Slight curving of extension member 16 will result in snugly securing tubular sleeve 20, thus maintaining it in any desired position.

An illumination housing base 22 is secured to an integral with tubular sleeve 20. An illumination housing cap 24 mates with illumination housing base 22, and is secured debt to by, for example, screws 26. Illumination is generated by a lamp 28, at one end of the housing formed by illumination housing base 22 and illumination housing cap 24. At the other end of the housing, a single pole single throw switch 30 forms of function of turning lamp 28 on and off.

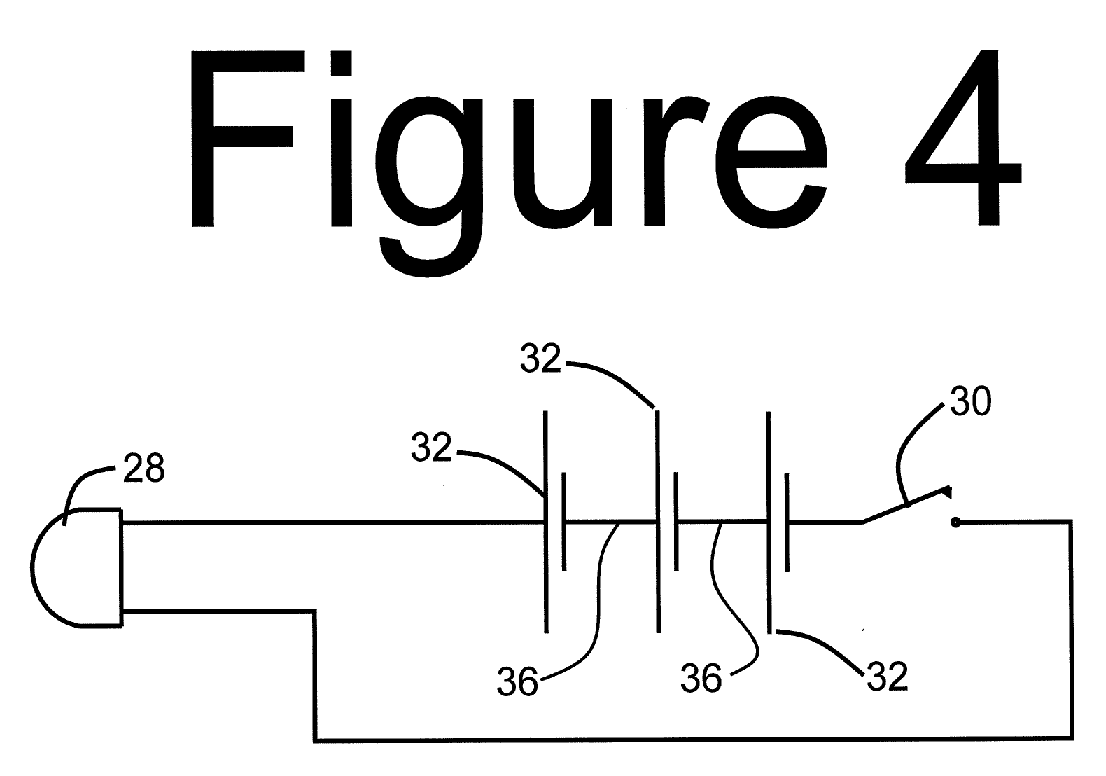

As illustrated in FIG. 4, lamp 28 is wired in series with switch 30 and batteries 32. Lamp 28 may take any of various forms, but in accordance with a preferred embodiment of the invention, a light emitting diode is used.

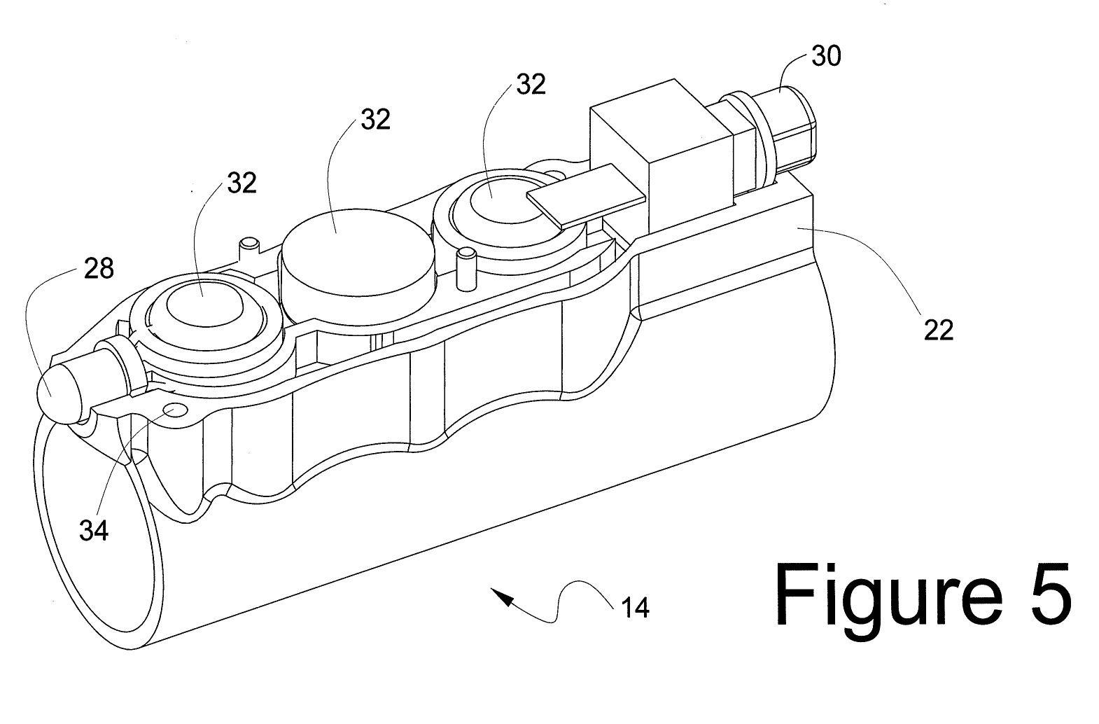

As can be seen in FIG. 5, when screws 26 are unscrewed, cap 24 may be removed, exposing batteries 32. When needed, batteries 32 may thus be replaced and cap 24 put back in place and secured with screws 26, which are received in holes 34.

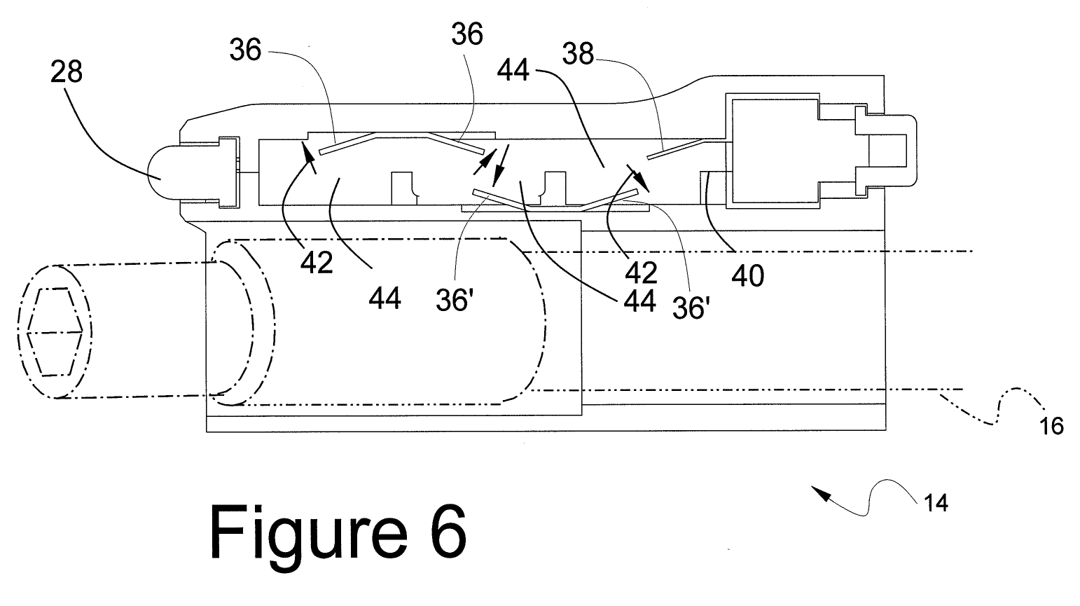

Referring to FIG. 6, illumination unit 14 is illustrated in cross-section. For purposes of understanding the function of the various members, in this view, batteries 32 have been removed, exposing leaf spring battery contacts 36 in their relaxed or un-deflected positions. As illustrated in FIG. 4, leaf spring contacts 36 function as wires in the circuit through which batteries 32 drive lamp 28.

More particularly, current flows through a path comprising switch lead 38, leaf spring contacts 36 lamp 28, leaf spring contacts 36′ and switch lead wire 40. That path forms a series circuit with voltages from batteries 32 being added to each other.

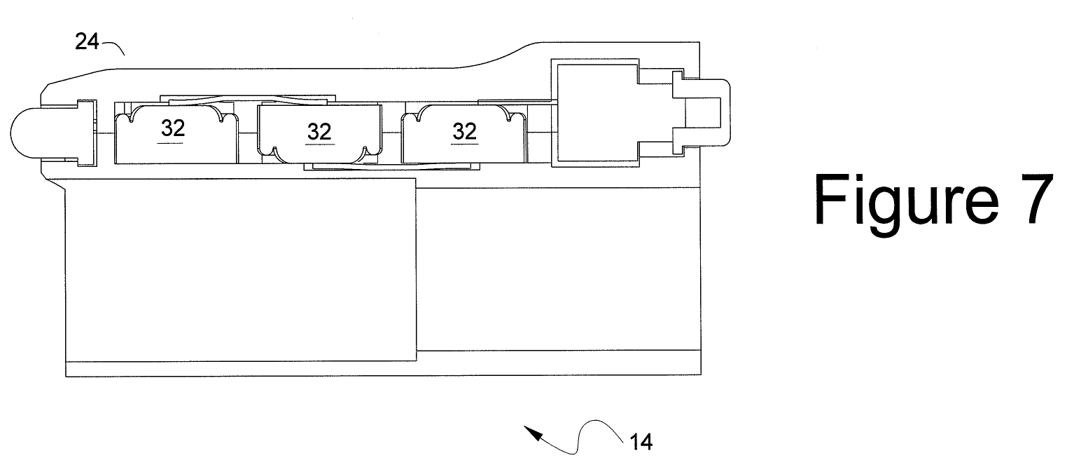

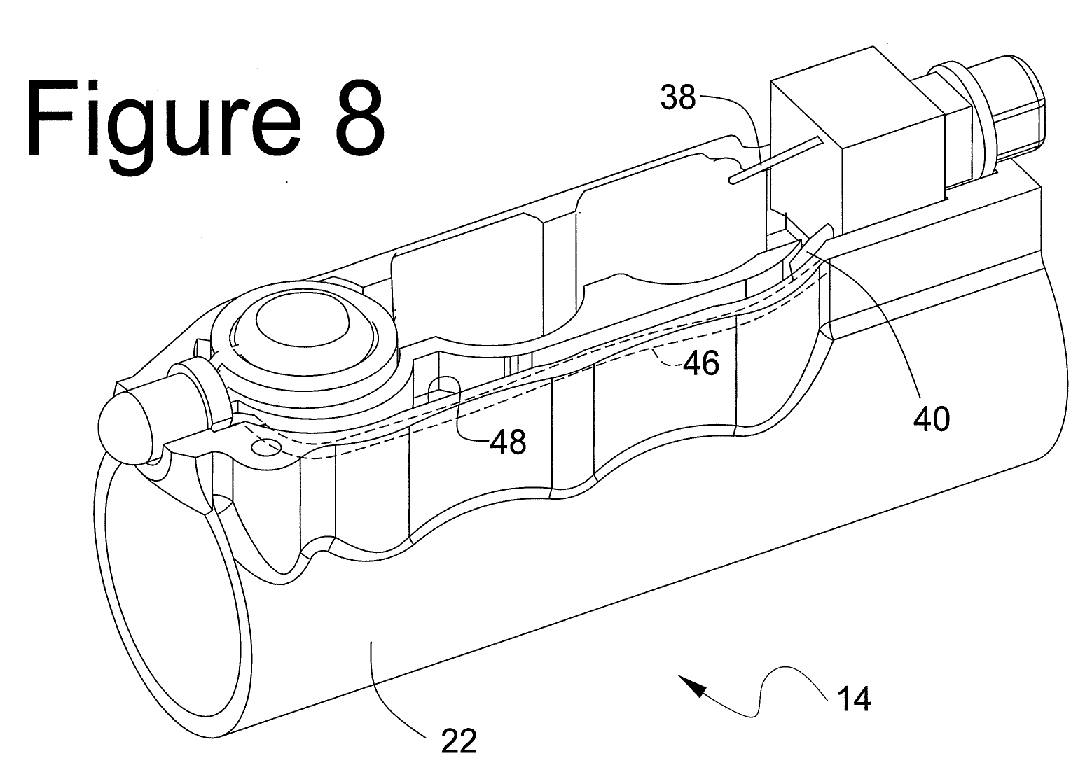

As illustrated in FIG. 7, when it is desired to insert batteries into the illumination unit 14, batteries are placed in volumes 44, causing the deflection of leaf spring contacts 36 in the directions indicated by arrows 42 in FIG. 6. Alluded to above illumination housing cap 24 may be removed from illumination housing base 22, resulting in exposure of volumes 44 to receive or accommodate replacement of batteries 32, as illustrated in FIG. 8.

As can be seen most easily in FIG. 8, light emitting diode lead 46 is positioned in a channel 48. When illumination housing cap 24 is brought into engagement with illumination housing base 22, light emitting diode lead 46 bears against lead 38 of switch 30.

In accordance with the invention, light emitting diode lamp 28 may be secured to illumination housing base 22. Likewise, switch 30 may be secured to illumination housing cap 24, for example by glue or a friction fit.

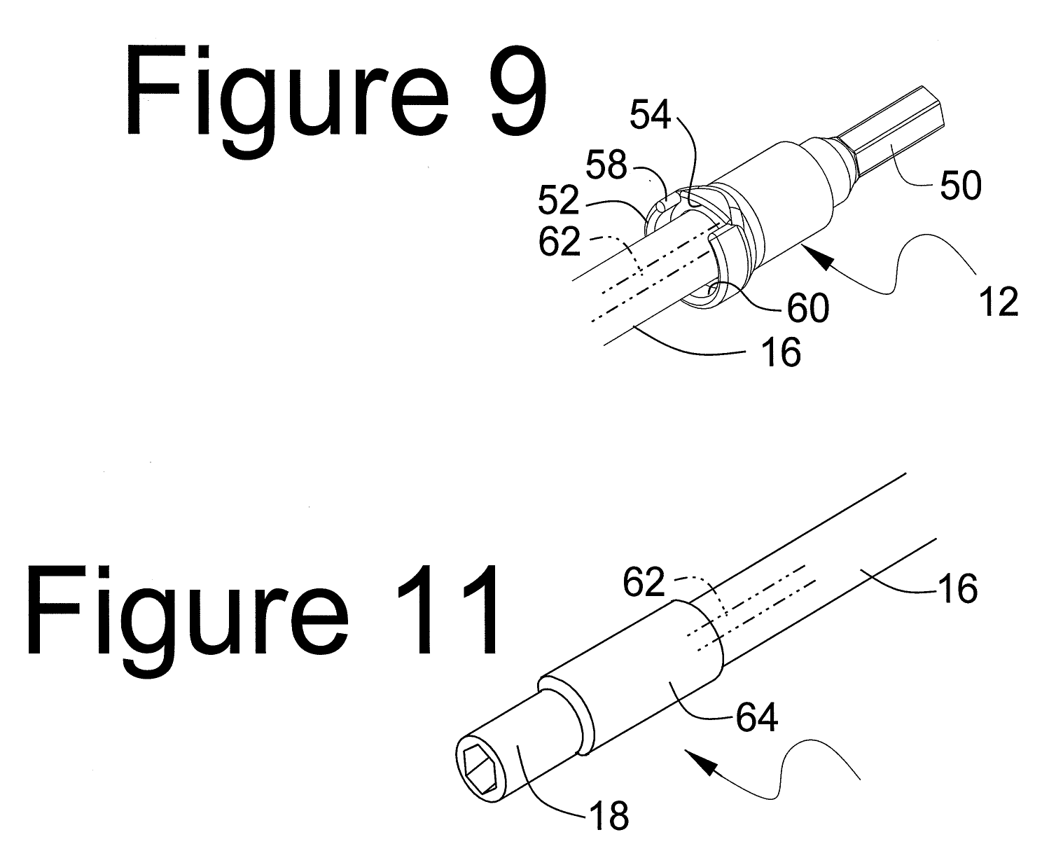

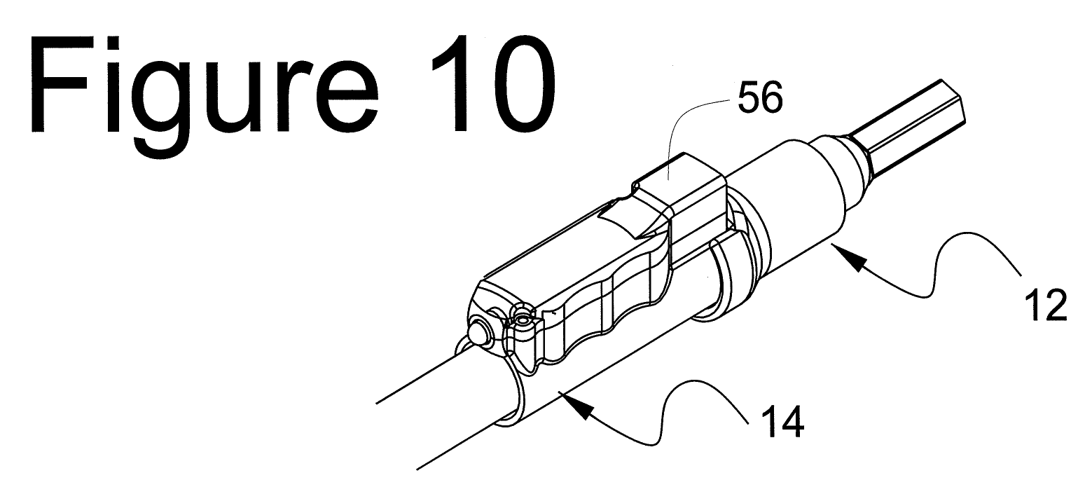

Referring to FIG. 9, base 12 comprises a hexagonal member 50 configured to mate with a standard hexagonal tool socket. Based 12 also includes a collar 52, which defines a cutaway portion 54. As illustrated in FIG. 10, cutaway portion 54 receives switch housing portion 56. Illumination unit 14 may be retained in the position illustrated in FIG. 10, by frictional engagement of switch housing portion 56 with the edges 58 of cutaway portion 54. Alternatively, frictional engagement with the inside surface 60 of collar 52 may be used to achieve the same result.

Referring back to FIG. 9, hexagonal shaft base 12 is secured to flexible extension member 16, for example by being crimped, welded or by any suitable means. In accordance with a preferred embodiment, hexagonal member 50 is secured to a central rotatable member 62, which rotates within flexible extension member 16, having a structure which is generally, per se, taught in the prior art. Likewise, in accordance with the present invention, it is contemplated that a variety of coupling structures and coupling members may be used at both ends of extension shaft 10.

Illumination unit 14 is able to slide from the position illustrated in FIGS. 2 and 10, to the position illustrated in FIGS. 1 and 3. In use, it would be in the forward position illustrated in FIG. 1, likely, for most users, most of the time. However, under certain circumstances, it may be desirable to vice illumination unit 14 as illustrated in FIG. 2. This would keep illumination unit 14 out of the way, for example in the event that the space where the work is being done is relatively cramped.

Referring to FIG. 11, an illustrative extension shaft chuck, comprising a hexagonal socket configured to receive standard hexagonal tool bits, such as Philips head tool bits, slot or standard screwdriver driver tool bits and drill tool bits.

In accordance with the invention, any suitable chuck may be used, such as a Jacobs™ type chuck or the like.

In accordance with the invention, collar 64 is secured to flexible extension member 16. Central rotatable member 62 is secured, at the end opposite the end of which is secured to base 12, to extension shaft chuck 18, which includes a hexagonal socket 66.

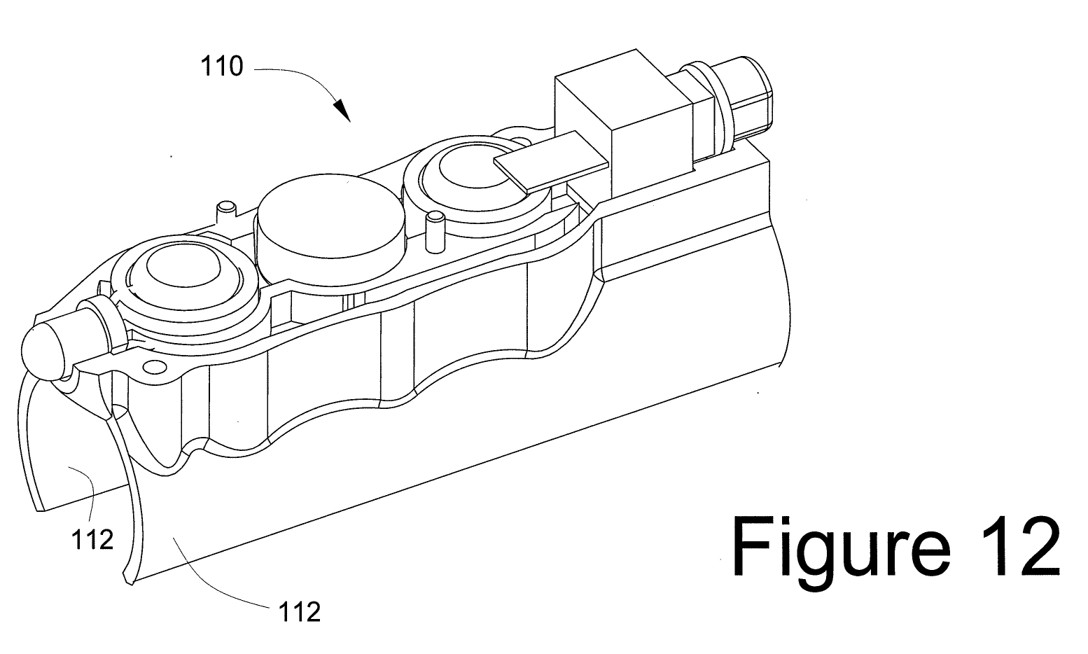

An alternative embodiment of a flexible extension shaft illumination unit 114 of the invention is illustrated in FIG. 12. In this embodiment, structures are substantially identical to those illustrated in the embodiment of Figures and 1-11, except that the cylindrical portion of illumination housing base 12 has been replaced by a pair of cylindrically contoured clamps 112.

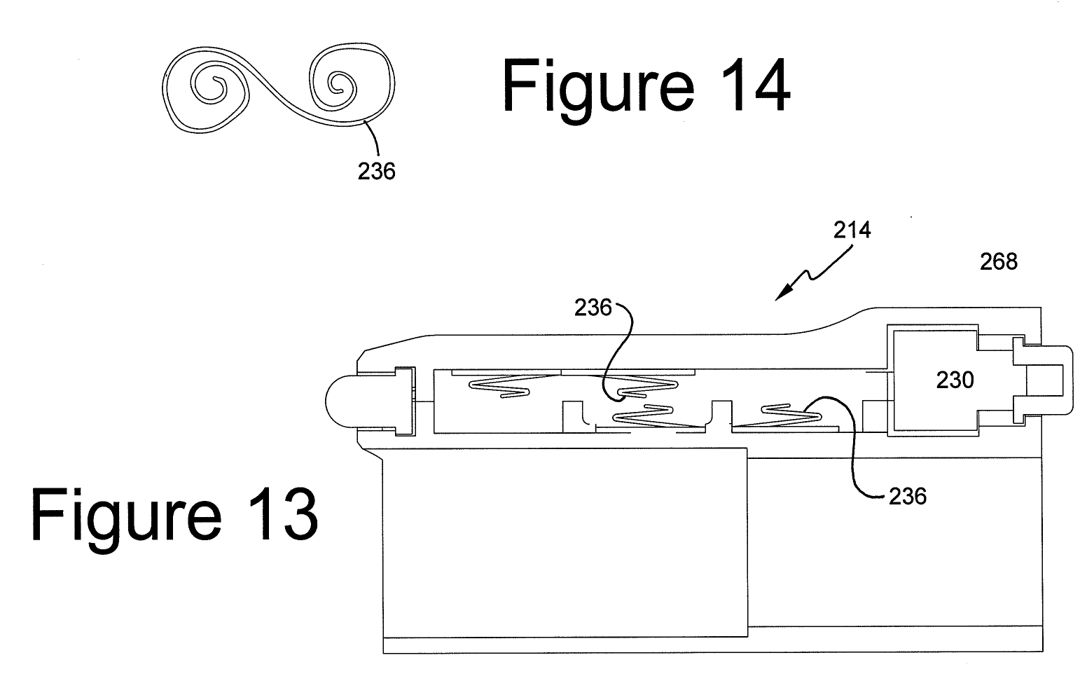

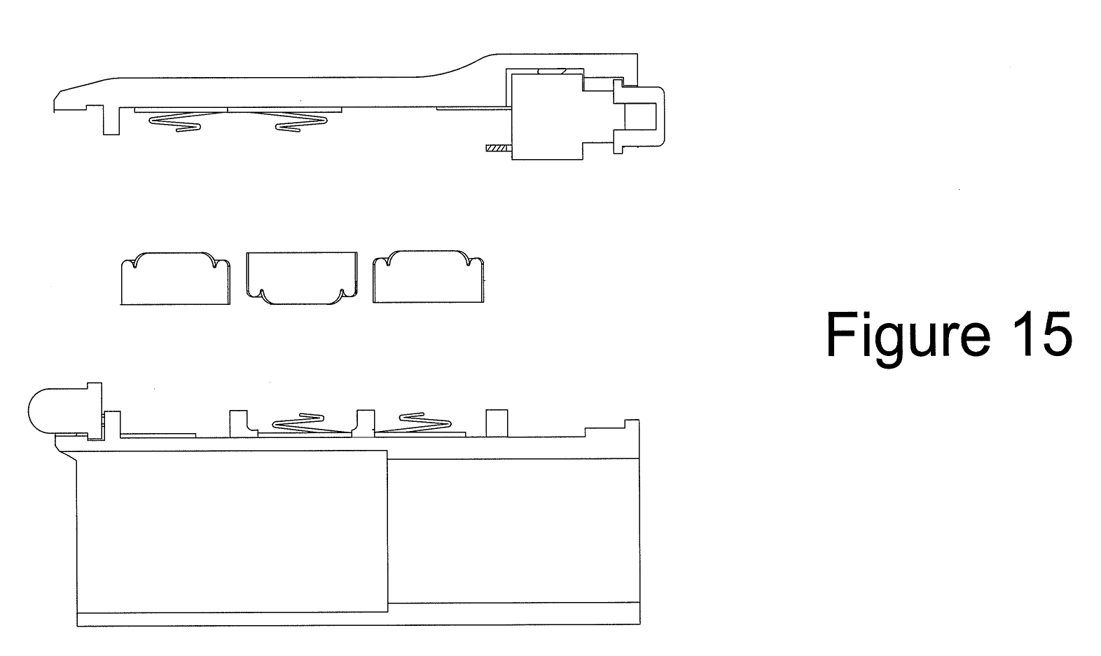

Referring to FIG. 13, another alternative embodiment of the invention is illustrated. In this embodiment, illumination unit 214 uses a double-coiled spring 236 in place of the leaf springs 36 of the earlier embodiments. Such a spring 236 is illustrated FIG. 14. In this embodiment, a switch 230 is secured by a quantity of glue 268. Referring to FIG. 15, the parts of illumination unit 214 are illustrated in exploded perspective.

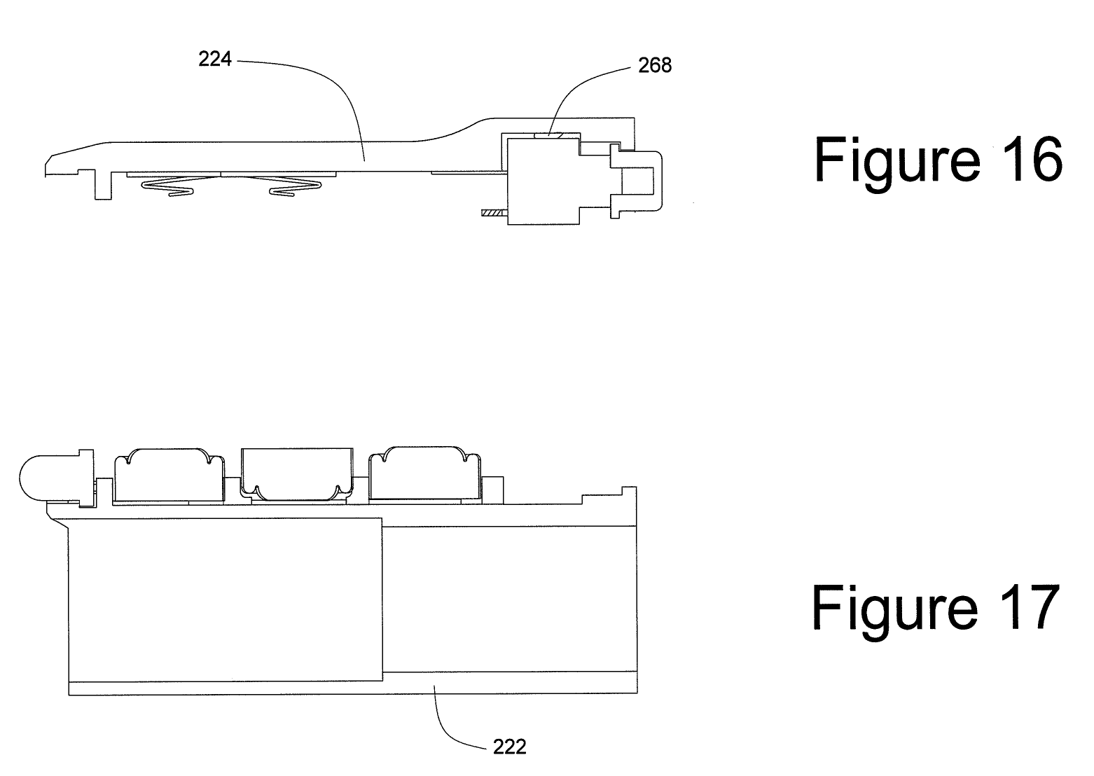

The replacement of batteries in accordance with the invention may be seen with reference to FIGS. 16 and 17. FIG. 16 schematically in cross-section illustrates the cap portion of the illumination unit in the embodiment of FIG. 12. FIG. 17 schematically in cross-section illustrates the base portion of the illumination unit in the embodiment of FIG. 12.

What is claimed:

1. A flexible extension shaft, comprising:
(a) a flexible extension member having a length, and one end and an opposite end;
(b) a tool base connection member secured to one end of said flexible extension member;
(c) a tool bit connection member secured to the opposite end of said flexible extension member; and
(d) a lighting unit secured to said flexible extension shaft.

2. Apparatus as in claim 1, wherein the lighting unit is slidingly mounted on said flexible extension member.

3. Apparatus as in claim 1, wherein said flexible extension member comprises an outer sleeve and a central rotatable member which rotates within said outer sleeve, said tool base connection member and said tool bit connection member being secured to said central rotatable member.

4. Apparatus as in claim 1, further comprising:
(e) a connection member securing said lighting unit to said flexible extension member.

5. Apparatus as in claim 4, wherein said connection member is a closed loop extending around said flexible extension member.

6. Apparatus as in claim 4, wherein said connection member is a clip.

7. Apparatus as in claim 1, wherein said lighting unit incorporates a switch.

8. Apparatus as in claim 1, wherein said lighting unit slides on said flexible extension member.

9. Apparatus as in claim 1, wherein said tool base connection member comprises a hexagonal bit for mating with the output of a power or hand tool and said tool bit connection member is a hexagonal socket chuck.

10. Lighting apparatus for illuminating a workpiece on a tool element comprising an extension shaft, said lighting apparatus comprising:
(a) a lighting unit; and
(b) a connection member securing said lighting unit to said flexible extension member.
